(19) United States
(12) Patent Application Publication
(10) Pub. No.: US 2019/0167675 A1
(43) Pub. Date: Jun. 6, 2019

(54) METHODS AND COMPOSITIONS FOR APPETITE CONTROL AND WEIGHT MANAGEMENT

(71) Applicant: SHANGHAI YAO YUAN BIOTECHNOLOGY CO., LTD., Shanghai (CN)

(72) Inventors: Jieqing FAN, Shanghai (CN); Yan LIU, Shanghai (CN); Cong XU, Shanghai (CN)

(73) Assignee: SHANGHAI YAO YUAN BIOTECHNOLOGY CO., LTD., Shanghai (CN)

(21) Appl. No.: 16/323,455

(22) PCT Filed: Aug. 9, 2016
(86) PCT No.: PCT/CN2016/094160

§ 371 Date: Feb. 5, 2019

(51) Int. Cl. A61K 31/4965; A61P 3/04; G01N 33/68

(52) U.S. Cl. CPC A61K 31/4965; G01N 33/6872; A61P 3/04

(57) ABSTRACT

The present invention provides methods and compositions for modulation of appetite, or for treatment of an appetite disorder or a metabolic disorder such as obesity or overweight, comprising a DEG/ENaC receptor modulator. The present invention also provides methods for identifying an agent for appetite modulation and/or weight management.

### TECHNICAL FIELD

The present invention generally relates to compositions and methods for appetite control and weight management, and for treating an appetite disorder and a metabolic disorder. In particular, the present application relates to use of agents modulating the expression or activity of DEG/ENaC ion channels for controlling appetite and treating disorders such as obesity, and also to methods of identifying potential new agents useful for controlling appetite and treating disorders such as obesity by assaying compounds which modulate the activity of DEG/ENaC ion channel.

### BACKGROUND OF INVENTION

Obesity/Overweight

In the recent decades, overweight and/or obese populations have been steadily rising worldwide, and particularly in the U.S. (Chaudhri, et al., 2005, Drug Discovery Today: Disease Mechanisms 2:289-294; Mokdad, et al., 2003, JAMA 289:76-79; Nguyen and El-Serag, 2 10, Gastroenterol Clin North Am 39:1-7; Wang and Beydoun, 2007, Epidemiol Rev. 29:6-28). Resulting from this is an alarming increase in diabetes, as well as other related health risks that have a significant impact on morbidity and quality of life. Not surprisingly, these consequential health risks incur substantial health and social costs (Kopelman, 2000, Nature 404:635-643; Must, et al., 1999, JAMA 282:1523-1529; Wang, et al., 2008, Obesity 16:2323-2330).

Obesity in China has also become a widespread disease. The etiology of obesity is multifaceted, ranging from genetic factors to environmental influences, such as the adoption of more sedentary lifestyles and the readily available sources of high-calorie food found in modern societies (Bleich, et al., 2008, Annu Rev Public Health 29:273-295; ROssner, 2002, Int J Obes Relat Metab Disord 26(Suppl 4):52-4). The exact mechanisms causing obesity, however, are still not clearly understood.

Currently there are 5 FDA approved anti-obesity drugs, including Xenical, a pancreatic lipase inhibitor, Qsymia, Belviq, and Contrave, agents suppressing appetite via effects on the central nervous system, and Saxenda, an agent acting on glucose metabolism. All these drugs lack strong efficacy (only 3-9% weight loss over 52 weeks) and cause serious side effects, including acute kidney injury, liver damage, headache, etc. Dropout rates for these drugs are up to 50%, mostly resulting from intolerable side-effects.

Worldwide demand for anti-obesity substances has led to research and study of drugs and foods that counteract the progressive body weight accumulation. Many agents involving different mechanism of action have been proposed for weight control, including drugs which can increase the motility of gastrointestinal tract, and drugs which can control appetite or sense of fullness by modulation of mechanosensation of gastrointestinal tract.

DEG/ENaC Ion Channels

The Degenerin/Epithelial Sodium Channel (Deg/ENaC) gene family encodes sodium channels involved in various cell functions in metazoans. This superfamily includes epithelial sodium channel (ENaC), acid-sensing ion channels (ASICs), pickpocket (PPK) genes in the Diptera order including Drosophila and mosquitoes, Degenerin subunits involved in sensory transduction in nematodes such as Caenorhabditis elegans, and peptide-gated Hydra Na+ channels (HyNaC) in hydrozoans (Israel Hanukoglu and Aaron Hanukoglu, Gene 579 (2016) 95-132).

Previous studies in Caenorhabditis elegans, Drosophila, and mice have shown that members of the Degenerin/Epithelial Sodium Channels function as a conserved family of mechanosensory ion channels (O'Hagan et al., 2005, Nature Neuroscience 8:43-50; Hwang et al., 2007, Current Biology 17:2105-2116; Zhong et al., 2010, Current Biology 20:429-434). A recent study (William H Olds1, Tian Xu, eLife 2014; 3:e04402) shows that enteric neurons play a major role in regulating feeding through specialized mechanosensory ion channels in Drosophila. Particularly, it has been found that PPK1 ion channels in Drosophila are present on posterior enteric neurons, which wrap around the muscles of the gut, and deficiency or pharmacological inhibition of the mechanosensory ion channel PPK1 gene result in an increase in food intake.

The mammalian members of the DEG/ENaC surperfamily are clearly distinct from their homologs in invertebrate Metazoan species in low sequence similarity. The mammalian DEG/ENaC family includes two groups, the epithelial sodium channels (ENaCs) and the acid sensitive ion channels (ASICs). ENaCs have a well-established role in Na+ reabsorption in the distal nephron, in the distal colon, and in the control of the liquid film on airway epithelia. ENaCs are inhibited by the drugs amiloride and triamterene that are clinically used as potassium sparing diuretics. ASICs are H⁺-activated channels found in central and peripheral neurons, where their activation induces neuronal depolarization. ASICs are involved in pain sensation, the expression of fear, and neurodegeneration after ischemia. There is no teaching in the prior art that the DEG/ENaC ion channels are involved in food intake or appetite control of mammal.

The inventors have found it desirable to overcome or ameliorate at least one of the disadvantages of the prior art, or to provide a useful alternative. In particular, the inventors have set themselves to create a therapeutic alternative for regulating appetite and weight management and for fighting overweight/obesity and obesity-associated disorders in mammal by modulation of the activity of DEG/ENaCs.

### SUMMARY OF THE INVENTION

In a first aspect, the present invention provides a method for regulating appetite by administrating a DEG/ENaC receptor modulator in a subject in need thereof, comprising administering to the subject a composition comprising a therapeutically effective amount of a modulator capable of modulating the activity of a DEG/ENaC receptor, and optionally a pharmaceutically acceptable carrier.

In a second aspect, the present invention provides a method of treating or preventing an appetite disorder or metabolic disorder such as obesity or overweight, or obesity-associated disorders in a subject, comprising administering to the subject of a composition comprising a therapeutically effective amount of a modulator capable of modulating the activity of a DEG/ENaC receptor, and optionally a pharmaceutically acceptable carrier.

In a third aspect, the present invention provides a method for identifying an agent for appetite modulation and/or weight management, said method comprising the steps of: providing an assay to determine modulation of expression or activity of an DEG/ENaC receptor; introducing to said assay a compound suspected of being an DEG/ENaC modulator; and determining whether DEG/ENaC modulation occurs, wherein the agent that modulates the level of expression or activity of the DEG/ENaC ion channel is a candidate for modulation of appetite or management of weight.

In a fourth aspect, the present invention provides a pharmaceutical composition for modulation of appetite or management of weight, or for treatment of an appetite disorder or metabolic disorder such as obesity or overweight or obesity-associated disorders, comprising: a DEG/ENaC receptor modulator and a pharmaceutically acceptable carrier.

For a complete understanding of the present invention and the advantages thereof, reference is made to the following detailed description of the invention. It should be appreciated that various aspects of the present invention are merely illustrative of the specific ways to make and use the present invention and do not limit the scope of the invention.

### BRIEF DESCRIPTION OF THE FIGURES

FIG. 1 shows phylogenetic tree of the epithelial sodium channel (ENaC)/degenerin (DEG) family. Protein sequences of ENaC, ASICs, and members representing other ENaC/DEG subfamilies, Drosophila pickpocket (PPK), the C. elegans DEG MEC4, and the peptide-gated FaNaC of H. aspersa were aligned by using the ClustalW algorithm. In addition, the bile acid-sensing ion channel, BASIC (also known as ASIC5, hINaC or BLINaC) is shown. The species are indicated with single letters, c, chicken; h, human; I, lamprey; r, rat; s, shark; t, toad fish; x, Xenopus; z, zebra fish. (Cited from Stephan Kellenberger and Laurent Schild, Pharmacol Rev 67:1-35, January 2015)

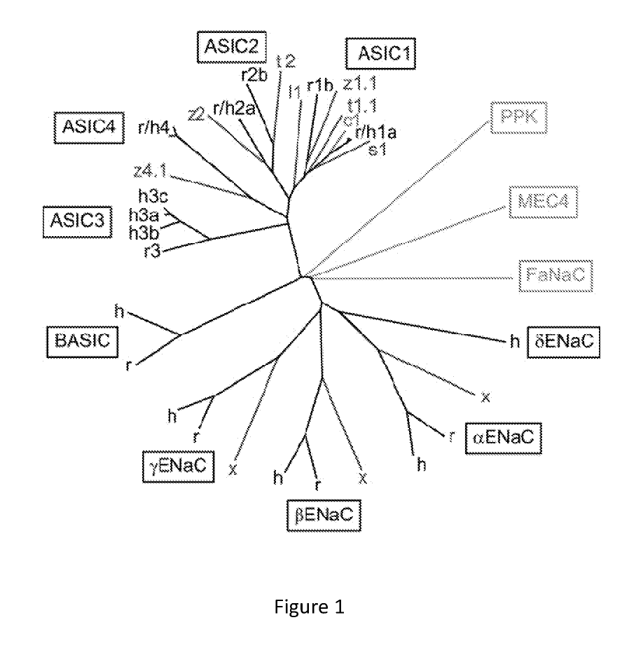

FIG. 2 shows the regulation of food intake by PPK1 ion channels in Drosophila posterior enteric neurons (PENs). (A) Outside and inside views of the hindgut (red, phalloidin, muscle) with posterior enteric neuron projections (green, 22C10). (B) PPK1 expresses in the PENs projecting to the hindgut pylorus (PPK1-Gal4; UAS-mCD8::GFP). (C) Food intake results for PPK1 deficiency (homozygote) and wild-type animals (heterozygote) (n=4-7 replicates). (D) Food intake results when PPK1 is inhibited using benzamil in wild-type (n=8-10 replicates). *=p<0.05, compared to corresponding DMSO controls.

FIG. 3 shows the expression of DEG/ENaC ion channels in gastrointestinal tract of mice. PCR reactions were performed using RNAs extracted from stomach, jejunum and colon from mice. The expressions of DEG/ENaC genes, αENaC, βENaC, ASIC1, ASIC2, ASIC3, ASIC5, and GADPH gene as control, were assessed.

FIG. 4 shows the structures of amloride and Benzamil. Benzamil is a more potent and specific antagonist of ENaCs.

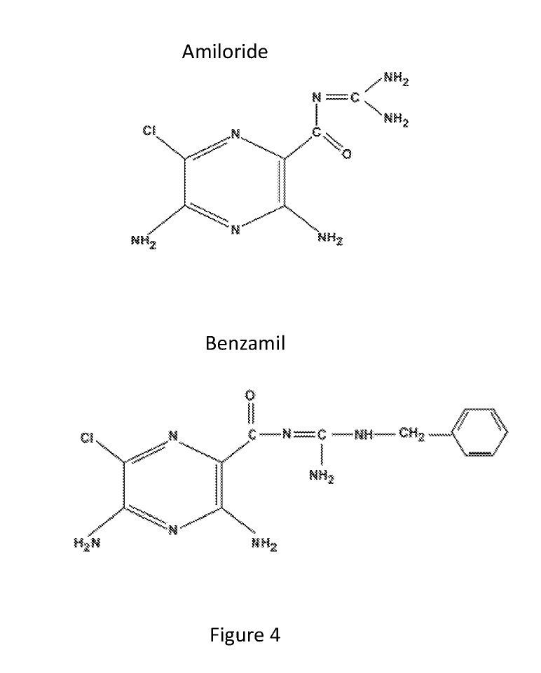

FIG. 5 shows the effect of amiloride on short-term food consumption in mice. (A) Female C57BL6 mice (Age 13 weeks old) were administrated via Oral gavage with 1, 10, or 100 μmole/kg amiloride (n=3 per concentration); (B) Male C57BL6 mice (Age 13 weeks old) were administrated via i.p. injection with 1, 10, or 100 μmole/kg amiloride (n=3 per concentration). The drug administration was made 15 minutes before night-time feeding from 6 μM. Food intake was monitored at the indicated times. The results are presented as the mean and standard error. *=p<0.05, and **=p<0.01, compared to corresponding vehicle controls.

FIG. 6 shows weight loss induced by amiloride in an obese animal. Obese model LepR^{PB} female mice were treated via oral gavage, with Amloride (n=10) or with vehicle DMSO (n=10) as control, 6 times a week for 5 weeks. Mice were weighed on Day 14, 21, 28 and 35. The weight change compared to Day 14 was plotted. Results presented as the mean and standard error. By day 35, mice fed with amiloride showed a clear reduction in weight compared with mice fed with DMSO (p<0.005).

FIG. 7 shows the change of body composition induced by amiloride in an obese animal. Mice were randomly assigned to receive amilorde (n=10) or DMSO (n=10). Before and after 5-week administration, mice were scanned by nuclear magnetic resonance (NMR) using a Bruker Minispec MQ10 NMR Analyzer to determine fat mass, lean mass, and free fluid. Compared to mice fed with DMSO, amiloride induced significantly more reduction in fat/lean ratio (p<0.05) and body fat percentage (p<0.02). But, no significant difference was noticed in reduction of body fluid percentage induced by amiloride and DMSO.

FIG. 8 shows the effect of Benzamil on short-term food consumption in mice. Female C57BL6 mice (Age 15 weeks old) were starved from 8 am to 6 μm, and then administrated via oral gavage, with 0.01, 0.1, 1, or 10 μmole/kg Benzamil (n=4 per concentration). Mice were fed with normal food 15 minutes after drug administration, and then food intake was monitored at the indicated times, 15 mins, 30 mins and 2 hrs after the start of feeding. The results are presented as the mean and standard error. Benzamil, when administrated immediately before feeding, induced reduction in short-term food consumption in mice.

### DETAILED DESCRIPTION OF THE INVENTION

The present invention is, at least in part, based on the finding that DEG/ENaC ion channel plays a role in regulation of food intake in a mammal, and inhibition of the DEG/ENaC ion channel can control appetite and thus induces loss of body weight in mammal.

Therefore, in one aspect, the present invention provides methods for regulating or controlling appetite by administrating a DEG/ENaC receptor modulator. In one embodiment, appetite may be suppressed to induce reduced food intake and/or loss of body weight. In another embodiment, appetite may be stimulated to induce an increase in food intake and/or body weight. In one embodiment, the modulator is administrated before or during food consumption, preferably before food consumption. In a further embodiment, the modulator is administrated 5 minutes to 3 hours before food consumption, for example, immediately before food consumption, such as 5-30 minutes. In some embodiments, the modulator induces fat loss in the subject.

In another aspect, the present invention provides methods for the treatment of an appetite disorder or a metabolic disorder in a subject in need thereof by administrating a DEG/ENaC receptor modulator. In one embodiment, the subject has an appetite disorder, such as overeating or undereating. In another embodiment, the subject has or is at the risk of having a disorder of appetite or a metabolic disorder such as obesity and/or obesity-associated disorder. In one embodiment, the modulator may induce weight loss and/or fat loss by suppressing appetite in a subject, preferably a subject suffering from obesity and obesity-associated disorder. In one embodiment, the modulator may stimulate appetite in a subject, preferably a subject suffering from a decreased desire to eat, to induce a desired weight gain.

In another aspect, the present invention provides a screening method for identifying new agents for appetite modulation and/or weight management or for the treatment of an appetite disorder or a metabolic disorder such as obesity and/or obesity-associated disorder, based on their ability of modulating a DEG/ENaC receptor.

In a further aspect, the present invention relates to pharmaceutical compositions comprising a DEG/ENaC receptor modulator and a pharmaceutically acceptable carrier for regulating appetite or managing weight, or for treatment of an appetite disorder, obesity and/or obesity-associated disorder.

### Definitions

The articles “a” and “an” are used herein to refer to one or to more than one (i.e. to at least one) of the grammatical object of the article. By way of example, “a DEG/ENaC protein” means one DEG/ENaC protein or more than one DEG/ENaC proteins.

As used herein, the words “comprise”, “comprises”, and “comprising” are to be interpreted inclusively rather than exclusively. Likewise, the terms “include”, “includes” and “including” should all be construed to be inclusive, unless such a construction is clearly prohibited from the context. Similarly, the term “examples,” particularly when followed by a listing of terms, is merely exemplary and illustrative and should not be deemed to be exclusive or comprehensive.

The term “about” will be understood by persons of ordinary skill in the art and will vary to some extent on the context in which it is used. Preferably, the term “about” is intended to modify a numerical value above and below the stated value by a variance of ≤20%, more preferably ≤10%.

“Overweight” is defined, for example, for an adult human as having a BMI between 25 and 30.

“Body mass index” or “BMI” means the ratio of weight in kg divided by the height in metres, squared.

“Obesity” is a condition in which the natural energy reserve, stored in the fatty tissue of animals, in particular humans and other mammals, is increased to a point where it is associated with certain health conditions or increased mortality. “Obesity” is defined, for example, for an adult human as having a BMI greater than 30.

As used herein, the term “treatment” or “treating” is defined as the application or administration of a therapeutic agent, i.e., a compound, such as a DEG/ENaC receptor modulator, e.g., amiloride, an analog or derivative thereof, useful within the invention (alone or in combination with another agent, for example, pharmaceutically acceptable carrier or adjuvant), to a subject, or application or administration of a therapeutic agent to an isolated tissue or cell either engineered or from a subject (e.g., for diagnosis or ex vivo applications), with the purpose to cure, heal, alleviate, relieve, alter, remedy, ameliorate, improve or affect the condition being treated, for example, an appetite disorder, overweight/obesity or obesity-associated disorder.

As used herein, the term “patient” or “subject” refers to a human or a non-human animal. Non-human animals include, for example, ovine, bovine, porcine, canine, feline and murine mammals. Preferably, the patient or subject is a mammal, and more preferably, a human.

DEG/ENaC Ion Channels

The mammalian members of the DEG/ENaC surperfamily include the epithelial sodium channels (ENaCs), and the acid sensitive ion channels (ASICs). Unless defined otherwise herein, the term “DEG/ENaC” used herein is intended to mean the mammalian DEG/ENaC members ENaCs and ASICs.

ENaCs are sodium channels, and are involved in salt homeostasis. The ENaC family is composed of four genes, SCNN1A, SCNN1B, SCNN1G, and SCNN1D, respectively encoding one of the four ENaC subunits alpha (human amino acid sequence database entry NP_001029.1 GI: 4506815 for isoform 1; NP_001153048.1 GI: 227430289 for isoform 2; NP_001153047.1 GI: 227430287 for isoform 3), beta (NP_000327.2 GI:124301196), gamma (NP_001030.2 GI: 42476333), and delta (NP_001123885.2 GI: 315259090). The gene for SCNN1D was not found in the mouse genome.

The ASICs are proton gated, non-selective cation channels, which are widely expressed in neurons of mammalian central and peripheral nervous systems. The ASIC family has been found to comprise discrete ASIC subunits: ASIC1 which has isoforms ASIC1a (human amino acid sequence database entry NP_064423.2 GI:21536351) and ASIC1b (NP_001086.2 GI:21536349) (also known as ASICα or BNaC2α and ASICβ or BNaC2B, respectively); ASIC2 which has isoforms ASIC2a (NP_899233.1 GI:34452695) and ASIC2b (NPJ301085.2 GI:9998944) (also known as MDEG1, BNaCI α or BNC1 and MDEG2 or BNACIβ, respectively); ASIC3 (NPJD04760.1 GI:4757710) (also known as DRASIC or TNaC); ASIC4 (NP_898843.1 GI:33942102) (also known as SPASIC); and ASIC5 (NP_059115.1 GI:74753059)(also known as BLINaC or hINaC, or BASIC).

ENaCs are assembled as a heteromultimer composed of α (or δ), β and γ subunits. Functional ASICs are thought to be composed of identical or different subunits (homo and heteromultimeric). The resolved structures of chicken ASIC1 revealed a homotrimer composed of three identical subunits. In DRG neurons, native ASICs are reported to be heteromultimeric. (Israel Hanukoglu and Aaron Hanukoglu, Gene 579 (2016) 95-132)

In one aspect, the methods and compositions of the present invention are useful for the modulation of the activity of a DEG/ENaC ion channel. In some embodiments, the DEG/ENaC ion channel is comprised of at least one subunit belonging to the mammal DEG/ENaC family. In some embodiments, the ion channel is comprised of three subunits selected from the group consisting of αENaC, βENaC, γENaC, δENaC, ASIC1a, ASIC1b, ASIC2a, ASIC2b, ASIC3, ASIC4, and ASIC5. In certain embodiments, the DEG/ENaC ion channel is a heteromeric ENaC protein composed of ENaC α, β, γ and δsubunits. In certain embodiments, the DEG/ENaC ion channel is an ASIC protein comprised of three subunit selected from the group consisting of ASIC1a, ASIC1b, ASIC2a, ASIC2b, ASIC3, ASIC4 and ASIC5.

In a further embodiment, the DEG/ENaC ion channel is amiloride-sensitive. The ENaCs and the ASICs form amiloride-sensitive ion channels. In some embodiments, the methods of the invention include modulation of the activity of an ENaC receptor and/or an ASIC receptor, more preferably one or more DEG/ENaC ion channels in gastrointestinal tract of the subject mammal. In a further embodiment, the methods of the invention include modulation of the activity of at least one DEG/ENaC protein selected from the group consisting of αENaC, βENaC, γENaC, ASIC1, ASIC2, ASIC3, ASIC4 and ASIC5.

DEG/ENaC Modulators

Modulators of mammalian DEG/ENaC family members, as used herein, are agents that modulate (including increase or reduce) the activity of one or more members of the mammalian DEG/ENaC family, that is, αENaC, βENaC, γENaC, δENaC, ASIC1a, ASIC1b, ASIC2a, ASIC2b, ASIC3, ASIC4 and ASIC5, among others. In some examples, the modulators (activators or inhibitors) may change (increase or reduce) the channel activity of one or more members, such as the ability of the members to flux sodium ions through cell membranes (into and/or out of cells).

The modulator may be compounds (small molecules of less than about 10 kDa, peptides, nucleic acids, lipids, etc.), complexes of two or more compounds, and/or mixtures, among others. The modulator also includes naturally occurring and synthetic ligands, antagonists, agonists, peptides, cyclic peptides, nucleic acids, antibodies, antisense molecules, siRNAs, ribozymes, small organic molecules and the like. In one embodiment, the modulator interacts with a DEG/ENaC receptor. In another embodiment, the modulator modulates the level of expression of a DEG/ENaC receptor, preferably an ENaC receptor and/or an ASIC receptor, in cells, preferable cells in the gastrointestinal tract. In a further embodiment, the modulator enhances or decreases the transcription or translation of a DEG/ENaC receptor. In a further embodiment, the modulator is selected from the group consisting of, for example, catalytic and inhibitory oligonucleotide molecules targeted against the gene(s) encoding a DEG/ENaC receptor, and inhibitors of DEG/ENaC receptor transcription or translation, such as antisense molecules, siRNAs, or ribozymes.

“Inhibitors” and “activators” of a DEG/ENaC ion channel, as used herein, refer to activating, or inhibitory molecules. “Inhibitors” are compounds that; e.g., partially, substantially, or completely block activity, decrease, prevent, delay activation, inactivate, desensitize, or down regulate the activity or expression of a DEG/ENaC protein, e.g., antagonists or blockers. “Activators” are compounds that increase, open, activate, facilitate, enhance activation, sensitize, agonize, or up regulate the activity or expression of a DEG/ENaC protein, e.g., agonists.

In some embodiments, the DEG/ENaC modulator is an inhibitor capable of inhibiting both an ENaC channel and an ASIC channel, for example, an amiloride or amiloride analogue. In some embodiments, the modulator may be specific to ENaCs or ASICs. For example, compound A-317567 is specific for inhibition of ASIC proteins. In some embodiments, the modulator may be specific within one of the DEG/ENaC families. For example, if specific within the ASIC family, the ASIC inhibitor may be capable of inhibiting one or more ASICs (e.g., ASIC1a only or ASIC1a plus ASIC1b only) to the substantial exclusion of the other ASICs. PcTx1 is a specific inhibitor targeting ASIC1a.

DEG/ENaC Inhibitors

In one embodiment, the DEG/ENac inhibitor of the invention interacts with a DEG/ENaC ion channel, more preferably one or more DEG/ENaC ion channels in gastrointestinal tract. In a further embodiment, the inhibition brings about a decrease in appetite and/or body weight.

In some embodiments, the inhibitor of the invention targets the amiloride sensitive DEG/ENaC ion channels mentioned above, and competes with amiloride as an inhibitor. In a preferable embodiment, the DEG/ENac inhibitor is amiloride or amiloride analogue such as benzamil. In one embodiment, amiloride is used in the methods and compositions of the invention. In one embodiment, benzamil is used in the methods and compositions of the invention.

Amiloride, 3,5-diamino-6-chloro-N-(diaminomethylidene)pyrazine-2-carboxamide, is a nonspecific blocker of ENaCs and ASICs, with IC₅₀ values of the order of 0.1 μM for ENaCαβγ and 10-100 μM for ASICs.

Amiloride has the following structural formula

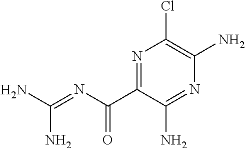 [SMILES: NC(N)=NC(=O)c1nc(Cl)c(N)nc1N]

Amiloride may be in any suitable nonionic form or ionic form (i.e., as a salt).

The term “amiloride analogue,” as used herein, means any structural analogue of amiloride, and more particularly, a chemical compound that is structurally related to amiloride and distinguished from amiloride by substitution at one or more positions. In some embodiments, an amiloride analogue is a compound of the following structural formula

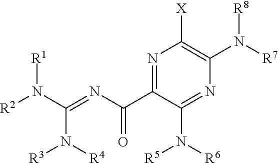

where X is halogen, such as fluoro, chloro, or bromo. In some embodiments, X is chloro. The amino substituents R¹-R⁸ may be selected independently from H, alkyl having 1-12 carbons, arylalkyl having 7-13 carbons, aryl, or heteroaryl. If one or more of substituents R¹-R⁸ is alkyl or arylalkyl, the alkyl portion of each alkyl or arylalkyl substituent may be optionally and independently further substituted one or more times by halogen, hydroxy, alkoxy having 1-6 carbons, aryl, heteroaryl, amino, alkylamino having 1-6 carbons, dialkylaminio having 2-12 carbons, carboxylic acid, or an ester formally derived from carboxylic acid and an alcohol having 1-6 carbons. If one or more of substituent R¹-R⁸ is aryl, arylalkyl, or heteroaryl, the aromatic portion of each aryl, arylalkyl, or heteroaryl substituent may be independently further substituted one or more times by halogen, alkyl having 1-6 carbons, amino, alkylamino having 1-6 carbons, dialkylamino having 2-12 carbons, carboxylic acid, or an ester formally derived from carboxylic acid and an alcohol having 1-6 carbons. In some embodiments, each of substituents R¹-R⁸ is independently selected from H, alkyl having 1-6 carbons, and arylalkyl, each of which may be further substituted as discussed above.

In some embodiments, the amiloride analogue is a compound of the following structural formula

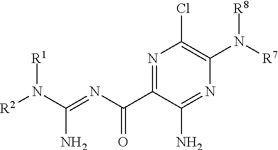

where R¹, R², R⁷ and R⁸ are independently H, alkyl having 1-6 carbons, or arylalkyl having 7-13 carbons.

In other embodiments, the inhibitor of the invention comprises an amiloride analog or a pharmaceutically acceptable salt thereof. In a related embodiment, the amiloride analog is selected from the group consisting of benzamil, phenmil, 5-(N-ethyl-N-isobutyl)-amiloride (EIPA), bepridil, KB-R7943, 5-(N-methyl-N-isobutyl) amiloride, 5-(N,N-hexamethylene) amiloride and 5-(N,N-dimenthyl) amiloride hydrochloride. In another related embodiment, the amiloride analog is benzamil. In another related embodiment, the amiloride analog is a methylated analog of benzamil. In another related embodiment, the amiloride analog comprises a ring formed on a guanidine group. In another related embodiment, the amiloride analog comprises an acylguanidino group. In another related embodiment, the amiloride analog comprises a water solubilizing group formed on a guanidine group, wherein the water solubilizing group is a N,N-dimethyl amino group or a sugar group.

In some embodiment, the inhibitor of the invention targets an amiloride sensitive DEG/ENaC protein, as described above, and competes with amiloride as a blocker. Known blockers include triamterene, phenamil, benzamil and derivatives thereof, particularly, 3′, 4′-dichlorobenzamil; 2′,4′-dimethylbenzamil; 5-(N-ethyl-N-isopropyl) amiloride; and 5-(N-methyl-N-isobutyl) amiloride.

Additional amiloride analogues and derivatives include the compounds described in Thomas R. et al. J. Membrane Biol. 105, 1-21 (1988); WO2012035158; WO2009074575; WO2011028740; WO2009150137; WO2011079087; and WO2008135557, each of which are herein specifically incorporated by reference.

In some embodiments, the subject is a human, and the amiloride, amiloride analog or a pharmaceutically acceptable salt thereof is given in a dose range of 0.01-3 mg/kg body weight/day in human. In some embodiments, the subject is a rodent, for example, a mouse, and the amiloride, amiloride analog or a pharmaceutically acceptable salt thereof is given in a dose range of about 0.1-40 mg/kg/day, for example, 0.12-37 mg/kg/day.

In some embodiments, ENaC inhibitors are used in the methods or composition of the present invention. An ENaC inhibitor may be any agent and/or composition capable of substantially reducing (including eliminating) the activity of at least one ENaC protein. An example of known ENaC blockers is triamterene, which specifically blocks γENaC, and is a potassium-sparing diuretic. Other examples of ENaC blockers include P301, P365, P321, P552-02, P1037, GS-9411/P680, which are developed by Parion Sciences (https://clinicaltrials.gov; http://www.parion.com/pipeline/p-1037-pulmonary-disease/). GS-9411/P680 from Parion Sciences/Gilead has been subject to Phase I to treat cystic fibrosis as an inhaled formulation (O'Riordan T G et al., Journal of Aerosol Medicine and Pulmonary Drug Delivery 2014, 27 (3): 200-8). P301 and P365 increase tear volume when applied to eyes (William R. Thelin, et al., J Ocul Pharmacol Ther. 2012 August; 28(4): 433-438); P321 is in Phase II for chronic dry eyes (http://www.parion.com/pipeline/p-321-dry-eye/). Another example of ENaC blockers is NVP-QBE170 from Novartis. NVP-QBE170 is a dimeric-amiloride derivative that shows a potent and selective blockage of ENaC both in vitro and in vivo. Its potency is similar to P552-02 from Parion Science but with a significantly enhanced safety window over existing ENaC blockers, in terms of hyperkalaemia, when tested in guinea pig TPD model. P552-02 and NVP-QBE170 are both amiloride analogs (K J Coote, et al., Br J Pharmacol. 2015 June; 172(11):2814-26.), and their chemical structures are as follows:

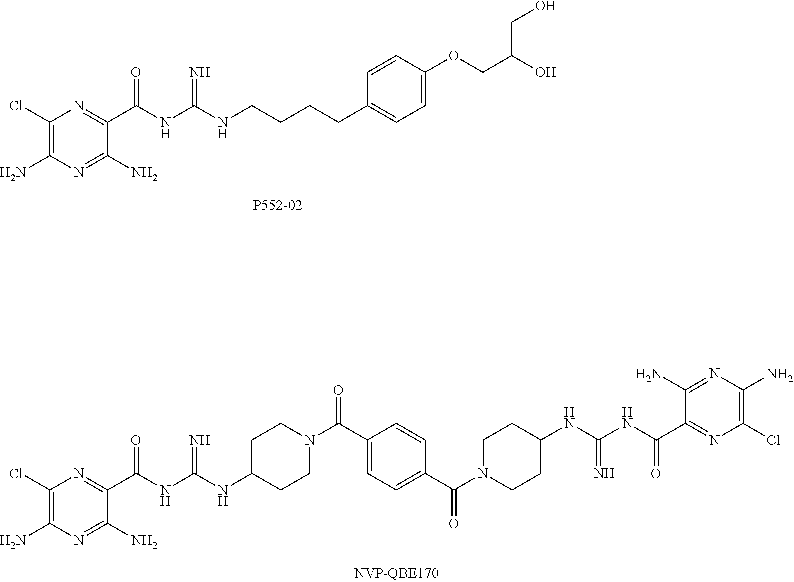 [SMILES: N=C(NCCCCc1ccc(OCC(O)CO)cc1)NC(=O)c1nc(Cl)c(N)nc1N ; N=C(NC(=O)c1nc(Cl)c(N)nc1N)NC1CCN(C(=O)c2ccc(C(=O)N3CCC(NC(=N)NC(=O)c4nc(Cl)c(N)nc4N)CC3)cc2)CC1]

In some embodiments, ASIC inhibitors are used in the methods or composition of the present invention. Examples of known ASIC blockers include amiloride, A-317567, A-317567 analogs, and aromatic diamidines.

A-317567 (CAS Regis. #: 371217-32-2, from Abbott Laboratories) is a small molecule non-amiloride blocker of ASIC having the following structural formula.

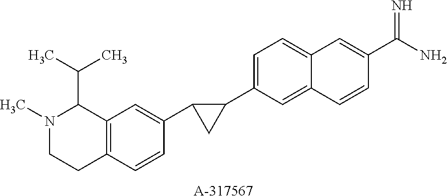 [SMILES: CC(C)C1c2cc(C3CC3c3ccc4cc(C(=N)N)ccc4c3)ccc2CCN1C ; "A-317567"]

The compound is peripherally active, and is 1.8-15 fold more potent than Amiloride to evoke ASICs currents in Rat DRG neurons (in vitro). Analgesic effect of A-317567 has been tested in CFA model of chronic inflammatory pain.

Scott D. Kuduk, et al. (ACS Chem Neurosci. 2010 Jan. 20; 1(1):19-24) reported A-317567 analogues, which are more potent than A-317567, especially compound ‘10a’ and ‘10b’ (about 3 fold)

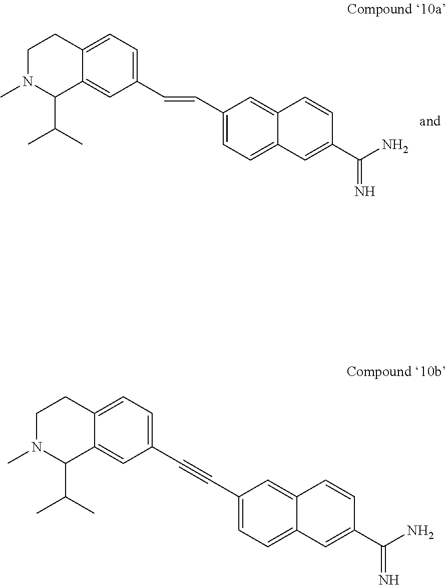 [SMILES: CC(C)C1c2cc(/C=C/c3ccc4cc(C(=N)N)ccc4c3)ccc2CCN1C ; CC(C)C1c2cc(C#Cc3ccc4cc(C(=N)N)ccc4c3)ccc2CCN1C]

Aromatic diamidines are synthetic small molecules that bind to the minor groove of DNA. They have been clinically used in the treatment of protozoan or fungus-infected diseases. Several anti-protozoal diarylamidines, 4′,6-diamidino-2-phenylindole (DAPI), diminazene, hydroxystilbamidine (HSB) and pentamidine, show potent ASICs blockage activity in vitro. (Chen X, et al., Neuropharmacology. 2010 June; 58(7):1045-53; Xuanmao Chen, et al., Eur J Pharmacol. 2010 Dec. 1; 648(1-3):15-23)

In addition, some toxin peptides are known as ASIC modulators. The examples of ASIC-targeting inhibitory toxins include the spider toxin Psalmotoxin1 (PcTx1), the sea anemone toxin APETx2, and the snake toxins Mambalgin-1-3. Those ASIC-targeting inhibitory toxins (PcTx1, 0.46 mg i.t. or 23 mg/kg; mambalgins, 2.2 mg i.t. and i.c.v. or 110 mg/kg; APETx2, 1.8 mg intraplantar; 0.9 mg intravenous; 2.7 mg i.t. or 135 mg/kg) never produce excitotoxicity, spasms, convulsions, motor paralysis, nor ataxia upon in vivo injections in mice (A. Baron et al. Toxicon 75 (2013) 187-204).

PcTx1 of the spider Psalmopoeus cambridgei inhibits homomeric ASIC1a and heteromeric ASIC1a/2b with nanomolar potency. The peptide has the sequence of

EDCIPKWKGCVNRHGDCCEGLECWKRRRSFEVCVPKTPKT

The toxin peptide may be used without substantial purification as part of venom from the tarantula species, may be purified from the venom, may be synthesized chemically, or may be biosynthesized by an engineered organism, among others. In addition, PcTX1 derivative may be used in accordance with the present invention. PcTX1 derivative is a peptide with a chemical structure formally related to PcTX1 and distinguished from PcTX1 by one or more amino acid substitutions, deletions, and/or insertions. The PcTX1 derivative is described in for example US Patent Application 20080242588 and WO/2015/026339.

DEG/ENaC Activators

In one embodiment, the modulator used in the method of the invention is an DEG/ENaC activator, which enhances the activity of a DEG/ENaC receptor to bring about increase in appetite and/or body weight. In a further embodiment, the DEG/ENaC activator is selected from the group consisting of a DEG/ENac stimulatory small molecular, peptide and mimetics thereof. In a further embodiment, the DEG/ENac activator is selected from the groups consisting of compound 53969, N,N,N-trimethyl-2-((4-methyl-2-((4-methyl-1H-indol-3-yl)thio)pentanoyl)oxy)ethanaminium iodide and N-(2-hydroxyethyl)-4-methyl-2-((4-methyl-1H-indol-3-yl)thio)pentanamide (from Senomyx Inc.), GMQ, AP301, and any analog or derivative thereof, and any combination.

Compound 53969, [N-(2-hydroxyethyl)-4-methyl-2-(4-methyl-1Hindol-3-ylthio) pentanamide], is a small molecule activator of human ENaC. The compound 53969 was recently reported to reversibly stimulate the human ENaC in heterologous cell expression systems. This compound acts on ENaC by increasing the channel open probability with an apparent affinity (EC₅₀) of 1 mM. See, for example, Stephan Kellenberger and Laurent Schild, International union of basic and clinical pharmacology. XCI. structure, function, and pharmacology of acid-sensing ion channels and the epithelial Na+ channel, J Clin Pharmacol. 2014 March; 54(3): 341-350. doi:10.1002/jcph.203.

AP301 is an ENaC activator (Stephan Kellenberger and Laurent Schild, Supra). It is a human TNF-α-derived peptide composed of 17 natural amino acids (^{˜}2 kD). The cyclic peptide was shown to activate ENaC by increasing its open probability in heterologous expression systems. Pulmonary administration of the TIP peptide has been shown in a variety of small animal models of acute lung injury (ALI) to substantially alleviate pulmonary permeability edema of various pathophysiological conditions. In the presence of AP301, amiloride-sensitive Na+ currents (via ENaC) in rat, dog, and pig AEC type II cells were increased by about 9-, 13-, and 16-fold, respectively, versus baseline conditions. AP301 is currently undergoing clinical trials on inhalation.

The synthetic compound 2-guanidine-4-methylquinazoline (GMQ) is an ASIC activator (Stephan Kellenberger and Laurent Schild, Supra).

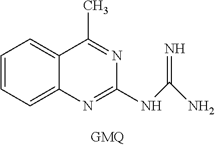

The compound GMQ induces persistent ASIC3 currents and induces pain related behavior.

Injection of the ASIC activator Mit-toxin (MitTx) of the Texas coral snake venom in the mouse paw induced pain behavior that was decreased by ASIC1a disruption (Bohlen C J, et al., (2011), Nature 479:410-414.).

Treatment

Appetite exists in all higher life-forms, and serves to regulate adequate energy intake to maintain metabolic needs. Abnormal appetite may cause malnutrition or overweight and metabolic disorders such as obesity and related problems. Health risks linked to obesity include heart disease and stroke; High blood pressure and high cholesterol; Diabetes; cancers for example cancers of the colon, breast (after menopause), endometrium (the lining of the uterus), kidney, and esophagus; Gallbladder disease and gallstones; Osteoarthritis; Gout; Breathing problems, such as sleep apnea (when a person stops breathing for short episodes during sleep) and asthma.

By showing the DEG/ENaC ion channels can be targeted pharmacologically to induce reduced food intake and weight loss, the present inventor proposed methods and compositions for appetite control and weight management in a subject. The methods and composition of the present invention avoids the side effects associated with current weight-control compounds for example anti-obesity drug that act directly on the brain.

In some embodiment, the methods and compositions may be used for the treatment of an appetite disorder and related disease, metabolic disorder or condition, including overweight, obesity and obesity-associated disorder, for example, diabetes type 2, hypertension, cardiovascular diseases, and combinations thereof.

In some embodiments, the methods and compositions may induce appetite suppression in a subject. In one embodiment, the subject is suffering from excessive appetite. In a further embodiment, the subject is suffering from obesity and/or overweight. In a further embodiment, the subject is suffering from obesity-associated disorder. In a further embodiment, the subject is benefit from the reduced food intake due to inhibition of a DEG/ENaC ion channel, for example, to maintain a desired weight or to get a desired loss of weight and loss of fat.

In some embodiments, the methods and compositions may induce appetite stimulation in a subject. In one embodiment, the subject is suffering from decreased appetite and/or weight loss associated with disorder such as cancer. In a further embodiment, the subject is benefit from an increase in food intake due to activation of a DEG/ENaC ion channel, for example, to get a desired gain of weight.

In one embodiment, the subject may be a human subject or a mammal animal subject that has overweight or obesity, or an obesity-associated disorder, and/or a significant chance of developing obesity or an obesity-associated disorder. Exemplary animals that may be suitable include any animal, such as rodents (mice, rats, etc.), dogs, cats, sheep, goats, non-human primates, etc. The animal may be treated for its own sake, e.g., for veterinary purposes (such as treatment of a pet). Alternatively, the animal may provide an animal model, for example an obesity mode, to facilitate testing drug candidates for human use, such as to determine the candidates' potency, window of effectiveness, side effects, etc.

Administration

The term “administer” or “administration” as used herein with respect to a drug or drug candidate and a subject, means to give or apply the drug or drug candidate to the subject such that the drug or drug candidate can exert its bioactive effect, if any, on the subject. Accordingly, administering a drug may include delivering the drug to a subject by any suitable route, including injection, ingestion, inhalation, topical application, or any combination thereof, among others. Injection may be performed subcutaneously, intradermally, intravenously, intra-arterially, intrathecally, epidurally, subdurally, intracerebroventricularly (i.e., into the brain), intraocularly, intraperitoneally, intra-synovially, or any combination thereof, among others. Injection may, for example, be via a needle or may be with a needle-free injector. Ingestion may be via a liquid formulation, a capsule, a tablet, or the like. Inhalation (or topical application to epithelia in the body) may be via an inhaler, atomizer, sprayer, or the like, and may involve a spray or particles/droplets of any suitable size, such as a spray or particles/droplets configured or sized for delivery to epithelia in the nose, mouth, pharynx, larynx, or lungs, among others. Topical application may involve placement of the drug onto an epithelial layer for trans-epithelial uptake. Exemplary epithelia for topical application may include external application to the skin or a wound thereof (i.e., direct placement onto the epidermis, dermis, hypodermis, or exposed wound tissue, among others). Other exemplary epithelia for topical application may include rectal, vaginal, urethral, oral, nasal, or ocular epithelia, or any combination thereof. Topical application may be facilitated by formulating the drug as an ointment and/or by placing the drug onto a dermal patch.

In some embodiments, the composition of the present invention is administered by a route selected from the group consisting of: orally, topically, sublingually, buccally, intranasally, rectally and intravenously. In some embodiments, amiloride or amiloride analog is administered orally or intravenously.

A therapeutically effective amount of a modulator (for example an inhibitor) may be administered. As used herein, the terms “effective amount,” “pharmaceutically effective amount” and “therapeutically effective amount” refer to a non-toxic but sufficient amount of an agent to provide the desired biological result. That result can be reduction and/or alleviation of the signs, symptoms, or causes of a disease, or any other desired alteration of a biological system, for example, the reduction of the body weight in an overweight or obese subject. An appropriate therapeutic amount in any individual case may be determined by one of ordinary skilled in the art using routine experimentation. For example, the effective amount of a DEG/ENaC modulator, oral amiloride, for an adult of about 75 kg is about 0.75-250 milligrams/day.

The regimen of administration may affect what constitutes an effective amount. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to produce a desired weight management, for example a reduced weight in an overweight or obese subject. An effective amount of the therapeutic compound necessary to achieve the desired effect may vary according to factors such as the age, sex, and weight of the subject. Dosage regimens may be adjusted to provide the optimum therapeutic response. For example, several divided doses may be administered daily or the dose may be proportionally reduced as indicated by the exigencies of the therapeutic situation. One of ordinary skill in the art would be able to study the relevant factors and make the determination regarding the effective amount of the therapeutic compound without undue experimentation.

In some embodiments, the subject is a human. In some embodiments, the modulator is amiloride, an amiloride analog or a salt thereof and is given at a daily dose (as a single dose or multiple dose) in the range of 0.01-30 mg/kg body weight, 0.01-10 mg/kg body weight, 0.01-5 mg/kg body weight, 0.01-3 mg/kg body weight, 0.01-2 mg/kg body weight, 0.01-1 mg/kg body weight, 0.01-0.3 mg/kg body weight, 0.01-0.1 mg/kg body weight, 0.01-0.03 mg/kg body weight, 0.03-30 mg/kg body weight, 0.03-10 mg/kg body weight, 0.03-5 mg/kg body weight, 0.03-3 mg/kg body weight, 0.03-1 mg/kg body weight, 0.03-0.3 mg/kg body weight, 0.03-0.1 mg/kg body weight, 0.1-30 mg/kg body weight, 0.1-10 mg/kg body weight, 0.1-3 mg/kg body weight, 0.1-1 mg/kg body weight, 0.1-0.3 mg/kg body weight, 0.3-30 mg/kg body weight, 0.3-10 mg/kg body weight, 0.3-3 mg/kg body weight, 0.3-1 mg/kg body weight, 1-30 mg/kg body weight, 1-10 mg/kg body weight, 1-3 mg/kg body weight, 3-30 mg/kg body weight, 3-10 mg/kg body weight or 10-30 mg/kg body weight. In one embodiment, the amiloride analog is selected from the group consisting of benzamil, phenamil, EIPA, bepridil, KB-R7943, 5-(N-methyl-N-isobutyl)-amiloride, 5-(N,N-hexamethylene)-amiloride, 5-(N,N-dimenthyl)amiloride hydrochloride, P552-02, and NVP-QBE170.

In other embodiments, the modulator is amiloride, an amiloride analog or a salt thereof and is administered as a pharmaceutical composition formulated as a single dose in the range of 0.1-1000 mg/dose, 0.1-300 mg/dose, 0.1-100 mg/dose, 0.1-30 mg/dose, 0.1-10 mg/dose, 0.1-3 mg/dose, 0.1-1 mg/dose, 0.1-0.3 mg/dose, 0.3-1000 mg/dose, 0.3-500 mg/dose, 0.3-300 mg/dose, 0.3-100 mg/dose, 0.3-30 mg/dose, 0.3-10 mg/dose, 0.3-3 mg/dose, 0.3-1 mg/dose, 1-1000 mg/dose, 1-300 mg/dose, 1-100 mg/dose, 1-30 mg/dose, 1-10 mg/dose, 1-3 mg/dose, 3-1000 mg/dose, 3-300 mg/dose, 3-100 mg/dose, 3-30 mg/dose, 3-10 mg/dose, 10-1000 mg/dose, 10-300 mg/dose, 10-100 mg/dose, 10-30 mg/dose, 30-1000 mg/dose, 30-300 mg/dose, 30-100 mg/dose, 100-1000 mg/dose, 100-300 mg/dose, or 300-1000 mg/dose. In one embodiment, the amiloride analog is selected from the group consisting of benzamil, phenamil, EIPA, bepridil, KB-R7943, 5-(N-methyl-N-isobutyl)-amiloride, 5-(N,N-hexamethylene)-amiloride, 5-(N,N-dimenthyl)amiloride hydrochloride, P552-02, and NVP-QBE170. In some embodiments, amiloride or amiloride analog is formulated for intravenous injection, or oral administration.

In preferable embodiments, the modulator in accordance with the invention is administrated before or during food consumption, preferably 5 minutes to 3 hours, for example 15 minutes before food consumption.

Pharmaceutical Composition

Another aspect of the present application relates to a pharmaceutical composition for regulating appetite or for treatment of appetite disorder and the related disease, metabolic disorder or condition, such as overweight, obesity, or obesity-associated disorder. The pharmaceutical composition comprises an effective amount of a DEG/ENaC modulator and a pharmaceutically acceptable carrier. In some embodiments, the pharmaceutical composition comprises an amiloride analog or a pharmaceutically acceptable salt thereof, wherein the amiloride analog is selected from the group consisting of benzamil, phenmil, EIPA bepridil, KB-7943, 5-(N-methyl-N-isobutyl) amiloride, 5-(N,N-hexamethylene) amiloride, 5-(N,N-dimenthyl) amiloride hydrochloride, P552-02, and NVP-QBE170.

The modulator in accordance with the present invention, for example the inhibitor, may be administered in any suitable form and in any suitable composition to subjects. In some examples, the modulator may be in the form of a pharmaceutically acceptable salt.

As used herein, the term “pharmaceutical composition” refers to a mixture of at least one compound useful within the invention with a pharmaceutically acceptable carrier, when appropriate. The pharmaceutical composition facilitates administration of the compound to a patient.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

In some embodiments, the pharmaceutical composition is formulated for oral application. In other embodiments, the pharmaceutical composition comprises amiloride and/or amiloride analog formulated for oral application. For oral application, particularly suitable are tablets, dragees, liquids, drops, suppositories, or capsules, caplets and gel caps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

In some embodiments, the pharmaceutical composition is formulated for intravenous injection. In other embodiments, the pharmaceutical composition comprises amiloride and/or amiloride analog formulated for intravenous injection. Pharmaceutical compositions suitable for injectable use include sterile aqueous solutions (where water soluble) or dispersions, and sterile powders for the extemporaneous preparation of sterile injectable solutions or dispersion. For intravenous administration, suitable carriers include physiological saline, bacteriostatic water, Cremophor EL™ (BASF, Parsippany, N.J.) or fluid to the extent that easy syringability exists. The injectable composition must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating amiloride and/or amiloride analog in the required amount in an appropriate solvent, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active, ingredient plus any additional desired ingredient from a previously sterile-filtered solution thereof.

Screen Method

In another aspect, the present invention provides a method for identifying new agents for regulating appetite or treating appetite disorder or metabolic disorder, especially obesity or overweight or obesity-associated disorder, based on their ability of modulating, for example, inhibiting or stimulating a DEG/ENaC receptor.

In one embodiment, a method is provided for screening an agent for capability to modulate food intake or appetite and/or manage weight, said method comprising the steps of:
providing an assay to determine modulation of expression or activity of an DEG/ENaC receptor;
introducing to said assay a compound suspected of being an DEG/ENaC modulator; and
determining whether DEG/ENaC modulation occurs,

wherein the agent that modulates the level of expression or activity of the DEG/ENaC ion channel is a candidate for modulation of food intake or appetite or management of weight.

In a further embodiment, said method comprising the steps of:

(i) contacting said agent with a DEG/ENaC receptor, and

(ii) detecting any change in the activity of said DEG/ENaC receptor.

Candidate Compounds

The compounds tested as modulators of ENaC and/or ASIC protein can be small organic molecule, or biological entity, such as protein, e.g., antibody or peptide, sugar, nucleic acid, e.g., a polynucleotide, oligonucleotide, siRNA, antisense oligonucleotide or ribozyme, lipid, fatty acid, etc., to be tested for the capacity to modulate the activity of a DEG/ENaC ion channel. The test compound can be in the form of a library of test compounds, such as a combinatorial or randomized library that provides a sufficient range of diversity. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis. Typically, test compounds will be small organic molecules, and peptides. In one embodiment, the compound is an amiloride analog.

Assays

A variety of assays, including in vitro and in vivo assays, including cell-based models, are available to assess the modulation of the activity or expression of a DEG/ENaC protein. See for example, U.S. Pat. No. 9,244,081 (describing screening process for ENaC modulators), and United States Patent Application 20080242588 (describing screening process for ASIC modulators). See also Andrew J. Hirsh, et al. J. Med. Chem. 2006, 49, 4098-4115 (describing design, synthesis, and structure-activity relationships of an ENaC blocker); G. R. Dube et al. Pain 117 (2005) 88-96 (describing in vitro and in vivo characterization of an ASIC blocker). Those documents are incorporated herein for reference.

Screening may involve any suitable assay system that measures interaction between DEG/ENaC proteins and the set of candidate modulator for example inhibitors. Exemplary assay systems may include assays performed biochemically (e.g., binding assays), with cells grown in culture (“cultured cells”), and/or with organisms, among others.

In some embodiments, such assays for modulator for example inhibitors and activators include, e.g., expressing ENaC and/or ASIC protein in vitro, in cells, cell extracts, or cell membranes, applying putative modulator compounds, and then determining the functional effects on activity.

In some embodiment, a high throughput binding assay is performed in which the DEG/ENaC protein is contacted with a potential modulator and incubated for a suitable amount of time. A wide variety of modulators can be used, as described above, including small organic molecules, peptides, antibodies, and DEG/ENaC ligand analogs.

A wide variety of assays can be used to identify DEG/ENaC-modulator binding, including labeled protein-protein binding assays, electrophoretic mobility shifts, immunoassays, enzymatic assays such as phosphorylation assays, and the like. In some cases, the binding of the candidate modulator is determined through the use of competitive binding assays, where interference with binding of a known ligand is measured in the presence of a potential modulator. Ligands for the DEG/ENaC family are known. Also amiloride is known to inhibit ENaC and ASIC function. In such assays the known ligand is bound first, and then the desired compound i.e., putative enhancer is added. After the DEG/ENaC protein is washed, interference with binding, either of the potential modulator or of the known ligand, is determined. Often, either the potential modulator or the known ligand is labeled.

Methods of assaying ion channel function include, for example, patch clamp techniques, two electrode voltage clamping, measurement of whole cell currents, and fluorescent imaging techniques that use ion-sensitive fluorescent dyes and ion flux assays, e.g., radiolabeled-ion flux assays or ion flux assays. In some embodiments, candidate compounds may be tested in short circuit current (ISC) assay, as described for example, in K J Coote, et al., Br J Pharmacol. 2015 June; 172(11):2814-26. In some embodiments, the compounds that modulate ASIC activity may be tested in the presence of the composition and the acid in a whole cell patch-clamp mode, as described in for example, in G. R. Dube, et al., Pain 117 (2005) 88-96.

In some embodiments, a cell-based assay system is used to measure the effect of each candidate modulator for example inhibitor on ion flux, such as sodium ion flux, or acid-sensitive ion flux, in the cells. In some embodiments, the ion flux is a flux of sodium. For example, sodium flux can be measured by assessment of the uptake of radiolabeled sodium. In some embodiments, the assay system uses cells expressing an DEG/ENaC family member, such as ENaCαβγ, ASIC Ia or ASIC2a, or two or more distinct sets of cells expressing two or more distinct DEG/ENaC family members, such as ENaCαβγ and a ASIC family member(s), to determine the selectivity of each modulator for example inhibitor for these family members. The cells may express each family member endogenously or through introduction of foreign nucleic acid. In some examples, the assay system may measure ion flux electrophysiologically (such as by patch clamp), using an ion-sensitive or membrane potential-sensitive dye (e.g., a sodium sensitive dye), or via a gene-based reporter system that is sensitive to changes in membrane potential and/or intracellular ion (e.g., sodium) concentrations, among others. The assay system may be used to test candidate modulator for selective and/or specific inhibition of DEG/ENaC family members, particularly ENaC ion channels and ASIC ion channels expressed in GI tract of mammal (for example human).

In some embodiment of the screen method, in the present or absence of the test compound, the modulation of the function of any cell expressing ENaC receptor(s) and/or ASIC receptor(s) are measured, including by way of example cells in the gastrointestinal tract such as enteroendocrine cells.

Samples or assays comprising ENaC and/or ASIC proteins that are treated with a potential modulator may be compared to control samples without the modulator, to examine the extent of modulation. Control samples (untreated with modulator) are assigned a relative protein activity value of 100%. In one embodiment, inhibition of ENaC or ASIC is achieved when the activity value relative to the control is about 80%, preferably 50%, more preferably 25-0%. In another embodiment, activation of ENaC or ASIC is achieved when the activity value relative to the control (untreated with activators) is 110%, more preferably 150%, more preferably 200-500% (i.e., two to five fold higher relative to the control), more preferably 1000-3000% higher.

Compounds identified in an in vitro assay, for example, a cell-based assay, and their biologically acceptable derivatives may be further tested in food intake or weight control tests using for example a normal mouse or obesity mouse model to confirm their effect on food intake or weight control.

It is to be understood that wherever values and ranges are provided herein, all values and ranges encompassed by these values and ranges, are meant to be encompassed within the scope of the present invention. Moreover, all values, in whole or partial increments, that fall within these ranges, as well as the upper or lower limits of a range of values, are also contemplated by the present application.

All patents, patent applications, publications, technical and/or scholarly articles, and other references cited or referred to herein are in their entirety incorporated herein by reference to the extent allowed by law.

### EXAMPLES

The invention is now described with reference to the following Examples. These Examples are provided for the purpose of illustration only, and the invention is not limited to these Examples, but rather encompasses all variations that are evident as a result of the teachings provided herein.

### Example 1

Regulation of Food Intake in Drosophila

The previous study in Drosophine indicates that the mechanosensory ion channel, PPK1, expresses in the posterior enteric neurons (PENs), and plays a role in regulation of food intake.

First, enteric neural projections were investigated in Drosophila using four previously characterized Gal4 fly lines by immunohistochemistry, using the following antibodies and fluorescent markers: rabbit Anti-GFP antibody (ab290; 1:1000; Abcam, Cambridge, UK), Alexa Fluor 488 Goat Anti-Rabbit IgG (H+L) (A11034; 1:800; Life Technologies, Gaithersburg, Md., USA), mAb22C10 (Developmental Studies Hybridoma Bank, University of Iowa), and Alexa Fluor 633 phalloidin (A22284; 1:250; Life Technologies). mAb22C10 is a microtubule associated protein highly expressed in axons, and thus can be labeled to show the morphology of the axons.

The expression of PPK1, a member of the DEG/ENaC superfamily, in the GI tract of Drosophine was examined using PPK1-Gal4 driving mCD8::GFP.

For the three-dimensional model of the posterior enteric neuron region, a z-stack series of confocal images were taken from a gut sample immunostained with mAb22C10 and Alexa Fluor 633 phalloidin and then converted into a model using Imaris. All images were acquired using a Zeiss LSM510 and analyzed using Imaris (Bitplane, Zurich, Switzerland).

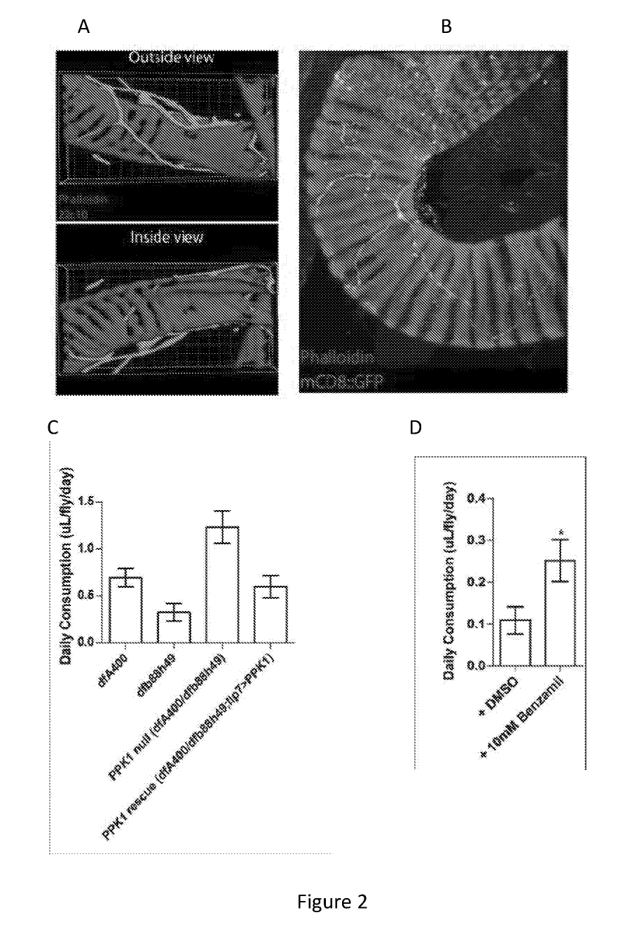

The results show posterior enteric neurons (PENs) tightly wrap around the muscles of the gut (FIG. 2A, PENs: green; muscles: red); and that PPK1 ion channels are present on the PENs (FIG. 2B).

Next, the effects of PPK1 deficiency and pharmacological inhibition on feed intake were examined in Drosophine. In short, flies were raised at 18° C. Capillary feeding assays were performed as described (Ja et al., 2007, Proceedings of the National Academy of Sciences of USA 104:8253-8256) on 2-day old males in groups of four at 29° C. for 24 hr. The diet was a 5% yeast extract and 5% sucrose solution. For the inhibition experiment, benzamil, an antagonist of DEG/ENaC ion channels, was used, and male yw flies were provided food with 100 mM sucrose supplemented with either 10 mM benzamil or DMSO.

PPK1 deficient flies had increased food intake (FIG. 2C). In consistent with the results, inhibition of PPK1 by benzamil resulted in increase in food consumption of Drosophine (FIG. 2D).

### Example 2

The members of DEG/ENaC superfamily in vertebrates share low sequence similarity with their homologs in invertebrates, and clearly represent different families. In mammal, there are two DEG/ENaC families, epithelial sodium channels (ENaCs) and Acid sensitive ion channels (ASICs). The ENaC family includes four ENaC homologs, ENaC α, β, γ, and δ. The ASIC family includes ASCI homologs, ASCI1a, ASCI1b, ASIC2a, ASIC2b, ASIC3, ASIC4, and ASIC5.

To investigate whether DEG/ENaC ion channels play a role in food intake in mammals, the following experiments were carried out.

### 2.1. Expression of DEG/ENaC Genes in Gastrointestinal Tract of Mice

Stomach, jejunum and colon were dissected from 8 weeks old C57B6 male mice. RNA was extracted using TRI reagent (invitrogen) and cDNA was prepared with PrimeScript reagent Kit (Takara). 10 ng RNA was used for each PCR reaction.

For RT-PCR, the following primers are used:

αENaC:
F:
5′-ACCTGTCGTTTCAACCAGGC
R:
5′-TCCAGGCATGGAAGACATCCAG
βENaC:
F:
5′-GGCCCAGGCTACACCTACA
R:
5′-AGCAGCGTAAGCAGGAACC
ASIC1:
F:
5′-ATGCTTCTCTCGTGCCACTTCC
R:
5′-TGGCCCGAGTTGAATGTGTAGC
ASIC2:
F:
5′-GCCCGCACAACTTCTCCTC
R:
5′-GGCAGGTACTCATCTTGCTGAA
ASIC3:
F:
5′-TTCGCTACTATGGGGAGTTCC
R:
5′-GCCATGTCAAAAGTCGGACTG
ASIC5:
F:
5′-CTGCCATCTCCAACTGACCG
R:
5′-CACCAAGAGCGAGACAGAGC

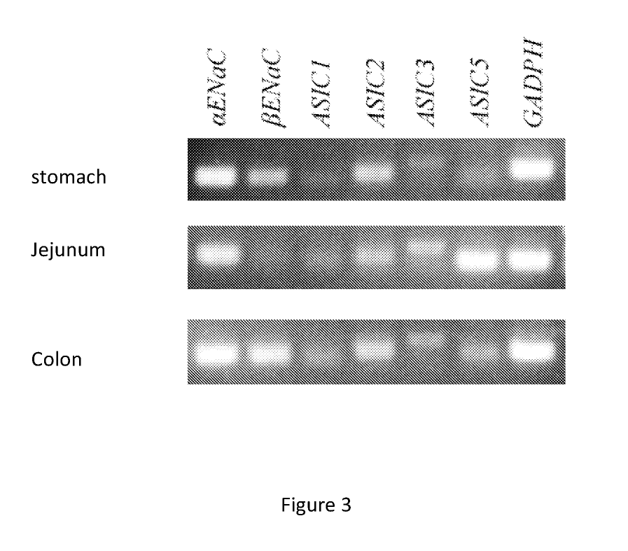

The DEG/ENaC genes tested, including αENaC, βENaC, ASIC1, ASIC2, ASIC3, ASIC5, were all expressed in stomach, jejunum and colon of the mice (FIG. 3). We hypothesized that mammal animals may have enteric neurons similar to the PENs in gut of Drosophila, which modulate food intake by the activity of DEG/ENaC ion channels present thereon.

### 2.2. Effect of Inhibition of DEG/ENaC Ion Channels on Food Intake

Amiloride is a known non-selective inhibitor of DEG/ENaC ion channels, which blocks ENaCs and ASCIs. The compound was first described by Cragoe et al. in 1967 (U.S. Pat. No. 3,313,813; Apr. 11, 1967; assigned to Merck Co., Inc.). The compound is used as an antihypertensive, potassium-sparing diuretic to treat hypertension and congestive heart failure. In hypertension patients, Amiloride works by inhibiting sodium reabsorption in the kidneys by binding to the amiloride-sensitive sodium channels. This promotes the loss of sodium and water from the body, but without depleting potassium.

In the following experiments, amiloride was used to antagonize DEG/ENaC ion channels in mice.

### 2.3. Suppression of Short-Term Food Intake in Normal Mice with Amiloride

13 weeks old C57BL6 Female and male mice were singly housed for 2 weeks before the experiment. Mice were starved Sam-6 μm. Then Amiloride was administrated by oral gavage or intraperitoneal injection, at 1, 10, or 100 μmole/kg body weight (229.6 μg, 2.296 mg, or 22.96 mg/kg body weight; n=3 animals per concentration), or vehicle (distilled water for oral gavage and saline for i.p. injection). The administrated volume was 10 ml/kg body weight. 15 minutes later, normal chow solid food was provided to the mice and the food consumption at designated time points were measured. P-values were calculated using t-test (unpaired, 2 tails) for data points. *=p<0.05, **=p<0.01, ***=p<0.001

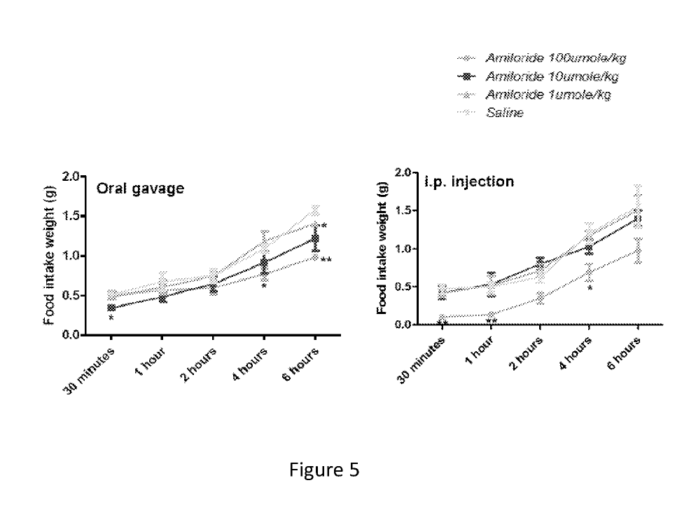

The results are shown in FIG. 5. Both oral and intraperitoneal administration of Amiloride suppressed short-term food intake in mice.

### 2.4. Weight Loss and Fat Loss in an Obese Mice Model with Amiloride

8 weeks old LepR^{PB} female mice were used in the experiment. LepR^{PB} mouse is a model of obesity, which carries a mutation in the gene for the leptin receptor. 20 mice were randomly divided into two groups. The mice in treatment group were administrated with amiloride 6 times a week via oral gavage in late afternoon and before nighttime feeding. Amiloride was dissolved in DMSO and diluted in sterile water. The dosage of amiloride administrated was respectively 4.1 mg/kg/day on Day 1-14, or 12.3 mg/kg/day on Day 15-35. The injection volume was 10 ml/kg. The mice in control group received 82 μl DMSO/kg/day in sterile water. Body weight was measured. The weight changes compared to the body weight on Day 14 were analyzed.

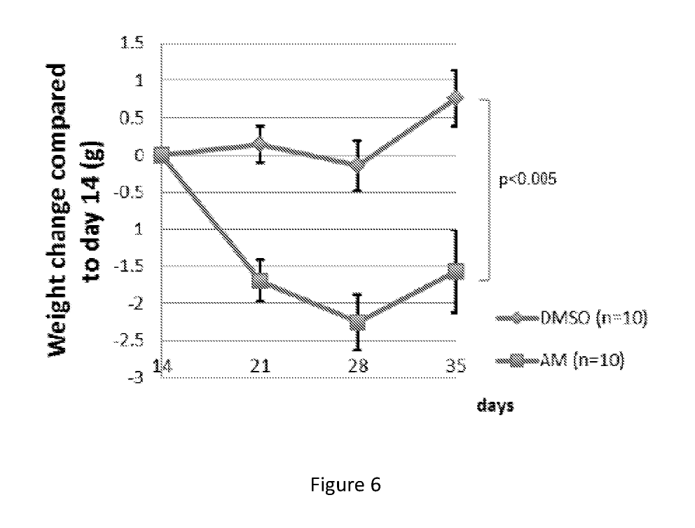

FIG. 6 shows the effect of amiloride on weight change of LepR^{PB} obese mice. The Leptin receptor mutant mice fed with amiloride showed significant reductions in body weight compared to control mice fed with DMSO. The data shows that amiloride has the effect of inducing weight loss.

### 2.5 Characterization of Amiloride Induced Weight Loss in Mice

To characterize the nature of the weight loss induced by amiloride, changes were determined in the body composition (including fat, lean and fluid) of the mice, before and after the 5 weeks drug administration, by Bruker Minispec LF50 NMR machine according to manufacturer's instructions.

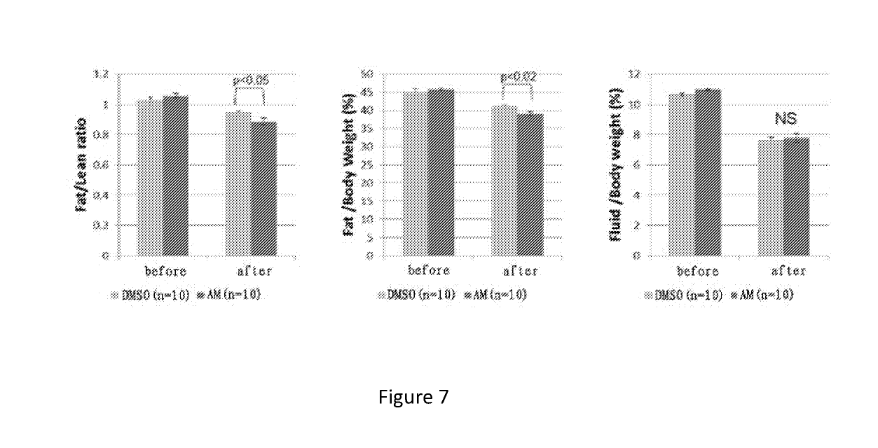

The results are shown in FIG. 7. Mice fed with amiloride and vehicle DMSO had a reduction in fat/lean ratio, body fat percentage, and body fluid percentage. However, amiloride resulted in significantly more reduction in fat/lean ratio and body fat percentage. The reduction in body fluid percentage of amiloride feeding mice was not significantly different compared to mice fed with control DMSO. The data suggests that amiloride induced weight loss is due to fat reduction than body fluid loss.

### 2.6. Suppression of Short-Term Food Intake in Normal Mice with Benzamil

To further determine whether the weight loss in mice fed with amiloride was due to inhibition of DEG/ENaC ion channels, an amiloride analogue, Benzamil, was used in a short-term food intake experiment. Benzamil is a more potent, highly specific and longer-acting antagonist of DEG/ENaC ion channels.

15 weeks old C57BL6 female mice were singly housed for 2 weeks before the experiment. Mice were starved Sam-6 μm. Then Benzamil (Benzamil hydrochloride hydrate) was administrated by intraperitoneal injection, 0.01-10 μmole/kg body weight (3.5621 μg 3.5621 mg/kg b.w.), or saline. The administrated volume was 10 ml/kg body weight. 15 minutes later, normal chow solid food was provided to the mice and the food consumption at designated time points were measured.

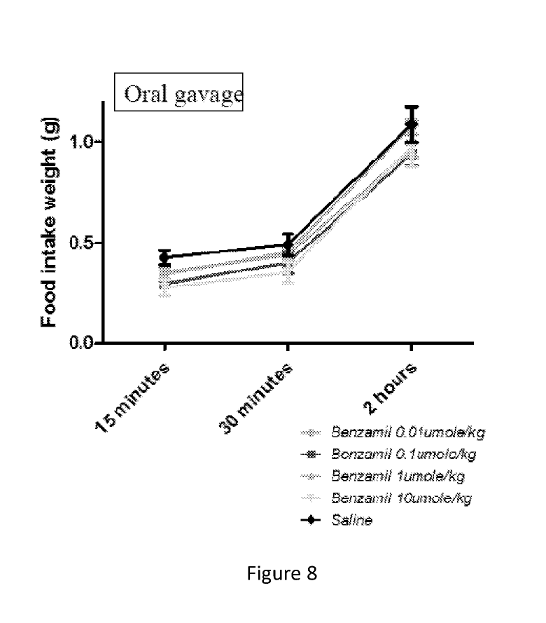

The results are shown in FIG. 8. Similar to amiloride, benzamilal suppressed short-term food intake in mice.

Taken together, our data supports the notion that the DEG/ENaC ion channels play a role in regulation of food intake in a mammal. However, unlike to the weight gain effect observed for the inhibition of its homolog PPK1 in Drosophi (Example 1), DEG/ENaC inhibition in mammal induces loss of body weight, which may be attributed to mainly body fat loss.

1. (canceled)

2. A method of regulating appetite, or treating or preventing an appetite disorder or a metabolic disorder, in a subject, comprising administering to the subject a therapeutically effective amount of a modulator capable of modulating the activity of a DEG/ENaC receptor.

3. The method according to claim 2, wherein the DEG/ENaC receptor is amiloride-sensitive.

4. (canceled)

5. (canceled)

6. The method according to claim 2, wherein the modulator is an inhibitor, and the appetite is suppressed to induce a reduced food intake and/or loss of body weight.

7. The method according to claim 6, wherein the modulator induces fat loss in the subject.

8. The method according to claim 2, wherein the modulator is an activator, and the appetite is stimulated to induce an increase in food intake and/or gain of body weight.

9. The method according to claim 2, wherein the modulator is administrated before or during food consumption.

10. The method according to claim 2, wherein the modulator interacts with a DEG/ENaC receptor in gastrointestinal tract.

11. The method according to claim 6, wherein the modulator comprises an inhibitor capable of inhibiting both an ENaC channel and an ASIC channel.

12. The method according to claim 6, wherein the modulator comprises amiloride or amiloride analog, or a compound of the following structural formula 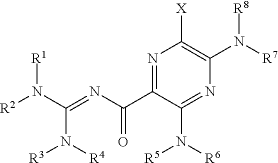
where X is halogen,
R¹-R⁸ is selected independently from H, alkyl having 1-12 carbons, arylalkyl having 7-13 carbons, aryl, or heteroaryl,
wherein the alkyl portion of each alkyl or arylalkyl substituent is optionally and independently further substituted one or more times by halogen, hydroxy, alkoxy having 1-6 carbons, aryl, heteroaryl, amino, alkylamino having 1-6 carbons, dialkylaminio having 2-12 carbons, carboxylic acid, or an ester formally derived from carboxylic acid and an alcohol having 1-6 carbons, and
wherein the aromatic portion of each aryl, arylalkyl, or heteroaryl substituent is optionally and independently further substituted one or more times by halogen, alkyl having 1-6 carbons, amino, alkylamino having 1-6 carbons, dialkylamino having 2-12 carbons, carboxylic acid, or an ester formally derived from carboxylic acid and an alcohol having 1-6 carbons.

13. The method according to claim 12, wherein the modulator is a compound of the following structural formula 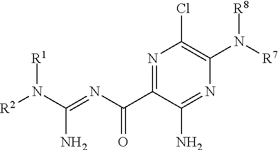
wherein R¹, R², R⁷ and R⁸ are independently H, alkyl having 1-6 carbons, or arylalkyl having 7-13 carbons.

14. The method according to claim 6, wherein the modulator comprises amiloride analog, selected from the group consisting of benzamil, phenamil, EIPA, bepridil, KB-R7943, 5-(N-methyl-N-isobutyl)-amiloride, 5-(N,N-hexamethylene)-amiloride, 5-(N,N-dimenthyl)amiloride hydrochloride, P552-02 and NVP-QBE170.

15. The method according to claim 12, wherein the modulator is administered in an amount of about 0.01-3 mg/kg body weight/day.

16. The method according to claim 6, wherein the DEG/ENac inhibitor comprises at least one selected from the groups consisting of Triamterene, A317567, A317567 analogs, P301, P365, GS-9411/P680, Aromatic diamidines, and any analog or derivative thereof, and any combination.

17. The method according to claim 8, wherein the DEG/ENac activator is selected from the groups consisting of compound S3969, N,N,N-trimethyl-2-((4-methyl-2-((4-methyl-1H-indol-3-yl)thio)pentanoyl)oxy)ethanaminium iodide and N-(2-hydroxyethyl)-4-methyl-2-((4-methyl-1H-indol-3-yl)thio) pentanamide, GMQ, AP301, and any analog or derivative thereof, and any combination.

18. The method according to claim 2, wherein the modulator comprises an ASIC-targeting modulator.

19. The method according to claim 2, wherein the DEG/ENac modulator modulates the level of expression or activity in gastrointestinal tract of a DEG/ENaC receptor.

20. The method according to claim 19, wherein the modulation is a decrease in the level of expression or activity, and the modulator is selected from the group consisting of catalytic and inhibitory oligonucleotide molecules targeted against the gene(s) encoding a DEG/ENaC receptor, and inhibitors of DEG/ENaC receptor transcription or translation.

21. (canceled)

22. A method for identifying an agent for appetite modulation and/or weight management, said method comprising the steps of:
providing an assay to determine modulation of expression or activity of an DEG/ENaC receptor;
introducing to said assay a compound suspected of being an DEG/ENaC modulator; and
determining whether DEG/ENaC modulation occurs,
wherein the agent that modulates the level of expression or activity of the DEG/ENaC ion channel is a candidate for modulation of appetite or management of weight.

23. A method according to claim 22, said method comprising the steps of:
(i) contacting said agent with a DEG/ENaC receptor, and
(ii) detecting any change in the activity of said DEG/ENaC receptor.

24. (canceled)

25. A pharmaceutical composition for modulation of appetite, or for treatment of an appetite disorder or a metabolic disorder, comprising:
a DEG/ENaC receptor modulator and a pharmaceutically acceptable carrier.
